(19) United States
(12) Patent Application Publication
(10) Pub. No.: US 2009/0259254 A1
(43) Pub. Date: Oct. 15, 2009

(54) Apparatus ans method for aligning and/or stabilizing the spine

(76) Inventor: Madhavan Pisharodi, Brownsville, TX (US)

Correspondence Address: Wisner & Associates;Suite 400, 1177 West Loop South, Houston, TX, 77027-9012, US

(21) Appl. No.: 12/148,006

(22) Filed: Apr. 15, 2008

(51) Int. Cl. A61B 17/70; A61B 17/04

(52) U.S. Cl. 606/246; 606/264; 606/301; 606/276

(57) ABSTRACT

Apparatus for use in stabilizing and/or aligning the spinal column. The apparatus utilizes hooks and rods to effectively transfer load from the vertebrae to the apparatus regardless of the angle and the height of the pedicular screws. The screws are retained to a screw cap that includes a hook forming a cradle for receiving the rod therein and a bore to which an extension is releasably secured. The extension includes a plurality of notches forming a ladder, each notch being formed so as to releasably retain the rod to the notch so that the rod can be moved down the ladder until it is positioned in the cradle of the screw cap, after which the extension is removed from the screw cap and a set screw is utilized to secure the rod in the cradle. The apparatus is also configured for transverse spinal fixation with rods that extend across the vertebral body to which longitudinal rods are affixed.

### BACKGROUND OF THE INVENTION

The present invention relates to an apparatus for aligning and/or stabilizing the spinal vertebrae. More specifically, the present invention relates to “hook and rod” external spinal stabilizers and/or alignment systems that facilitate alignment of the vertebrae using such stabilizers and/or systems.

Stabilization and/or alignment of the spinal column is indicated for treatment of many pathologies including trauma, tumor, infection, degenerative spine disease, iatrogenic spinal instability, spondylolysis/spondylolisthesis, pseudarthrosis, kyphosis, and spinal deformity. A number of stabilizers for treating such pathologies are disclosed in the scientific and patent literature, and are available commercially from such vendors as Sofamor-Danek, Osteonics, and others, that utilize so-called “hook and rod” components. Such stabilizers utilize screws that are driven into the bodies of adjacent vertebrae, typically in the pedicular region of the vertebral body, and rods that are secured to two, three, or more screws on adjacent segments using hooks that are either formed on or attached to the heads of the screws to stabilize and/or align the vertebrae.

A problem that arises during surgery utilizing such systems involves the point at which the screw is attached to the rod. Depending upon the location on the spine, size of the vertebra, pathology, and other factors unique to the patient, the screw heads extend at different angles and heights relative to the longitudinal axis of the spine even on adjacent vertebrae. For this reason, it can be difficult for the surgeon to attach the heads of the screws to the rod, particularly when the patient suffers from spinal deformity. When used to stabilize the spine, the rod is bent before it is attached to the screw, but when utilized to correct spinal deformity, the screw heads must be moved to the rod instead of forming the rod to the screws such that the connection between screw and rod must sometimes be accomplished using brute force. Of course the screw heads are being attached to the rods in a surgical stage populated with nerves and other tissues to which the application of force is anathemic. There is, therefore, a need for improvement in such instrumentation, and it is an object of the present invention to provide such improvements.

Another object of the present invention is to provide an apparatus for use in aligning the spinal column that reduces the likelihood of spinal trauma during surgery.

Similarly, in large part because of the difficult geometry caused by the different angles and heights of the pedicular screws, the transfer of the load that is normally carried by the spinal column to the stabilizer through the connection between the rod and the screw is problematical. Not only must this connection effectively transfer the load from the vertebrae to the spinal stabilizer, but the transfer must be accomplished at any of a plurality of angles between the pedicular screws and the stabilizer. Several prior art devices that appear to attempt to address this problem are the pedicular screw and hook believed to be marketed under the trademark ISOBAR by Scient'x, Maitland, Fla. (www.scientxusa.com). However, this prior art device does not appear to lock to the traverse or longitudinal bar to which it is hooked, nor does it include structure that facilitates attachment of the hook to the rod when pedicular screws do not line up and/or are at different heights.

It is therefore also an object of the present invention to provide an apparatus and method for effectively transferring load from the spinal column to a spinal stabilizer, regardless of the angle and height of the pedicular screws that anchor the stabilizer to the patient's spine.

It is also an object of the present invention to provide a method that facilitates the attachment of the rod of a spinal stabilizer by gradually working, or re-positioning, the rod in successive positions, or notches forming a ladder, until the rod is positioned so as to allow attachment of the rod to the pedicle screws.

Another object of the present invention is to provide structure on the spinal stabilizer that facilitates re-positioning of the rod from the successive position to the point at which the rod is attached to the pedicle screw.

There is also a need for spinal instrumentation for use in surgical treatment of spinal deformity and pathology that facilitates adjustment of the instrumentation to the individual patient and to the geometry and configuration of the patient's anatomy, and it is an object of the present invention to provide apparatus and methods meeting that need.

There is also a need for spinal instrumentation that is adaptable for use as both a lateral and a transverse spinal stabilizer as needed for treatment of the patient. There is also a need for spinal instrumentation for use in surgical treatment of spinal deformity and pathology that minimizes surgical trauma and it is also an object of the present invention to provide apparatus and methods meeting that need.

Another object of the present invention to provide an apparatus for use in aligning the spinal column having a construction that can be implanted quickly, shortening the time of the surgical procedure.

It is also an object of the present invention to provide an apparatus in which the rod of the spinal stabilizer can be locked against movement relative to the pedicle screw even when the rod is not attached to the screw at a right angle.

Other objects, and the many advantages of the present invention, will be made clear to those skilled in the art in the following detailed description of several preferred embodiments of the present invention and the drawings appended hereto. Those skilled in the art will recognize, however, that the embodiments of the invention described herein are only examples provided for the purpose of describing the making and using of the present invention and that they are not the only embodiments of spinal stabilizers that are constructed in accordance with the teachings of the present invention.

### SUMMARY OF THE INVENTION

The present invention addresses the above-described problem by providing an apparatus for use in aligning and/or stabilizing the spinal column comprising a screw comprised of an elongate shank and a head and a screw cap comprised of a base having a hook extending therefrom, the hook being adapted for retaining a rod to the screw cap. Means is formed in the base of the screw cap for retaining the screw cap to the head of the screw at any of a plurality of angles relative to the longitudinal axis of the screw. The invention also comprises an extension having a plurality of notches formed thereon, each of the notches being adapted for releasably retaining a rod to the extension having means formed thereon for releasably securing the extension to the screw cap.

In another aspect, the present invention provides a method of aligning the spinal column comprising the steps of:
driving a plurality of bone screws, the head of each bone screw being provided with a screw cap comprised of a base and a hook, into the bodies of a like number of spinal vertebrae;
affixing an extension to each of the bone screws, each extension having a plurality of notches adapted for releasably retaining a rod formed thereon;
inserting a rod running substantially parallel to the spinal column into a notch on each extension;
pushing the rod out of the notch and downwardly toward the head of the bone screw either into a next lower notch or until the rod is positioned under the hook of the screw cap; and
removing the extension from the screw cap.
   In a particularly preferred embodiment, the method of the present invention also contemplates securing the rod under the hook of the screw cap.

### BRIEF DESCRIPTION OF THE DRAWINGS

Referring now to the figures, FIG. 1 shows a perspective view of a first embodiment of an apparatus for use in aligning and/or stabilizing the spinal column constructed in accordance with the teachings of the present invention.

FIG. 2 is an elevational view of a bone screw and screw cap for use in connection with the apparatus of FIG. 1.

FIG. 3 is an elevational view of an extension for use in connection with the apparatus of FIG. 1.

FIG. 4 is an elevational view of the screw cap of FIG. 2.

FIG. 5 is a sectional view of the apparatus shown in FIG. 1, taken along the line 5-5 in FIG. 1.

FIG. 6 is a sectional view of an alternative embodiment of the screw and screw cap shown in FIG. 5.

FIG. 7 is a sectional view of the apparatus shown in FIG. 1, taken along the line 7-7 in FIG. 1.

FIGS. 8A, 8B, and 8C are perspective, schematic views of the apparatus of FIG. 1 showing successive steps in the use of the apparatus in a first embodiment of a method for aligning a spinal column in accordance with the teachings of the present invention.

FIG. 9 is a schematic, perspective view of the apparatus of FIG. 1 as configured for use as a transverse spinal stabilizer.

FIG. 10 is a perspective view of the connector of the transverse spinal stabilizer of FIG. 9 that has been removed therefrom.

FIG. 11 is a sectional view, taken along the lines 11-11 in FIG. 9, of the connector of FIG. 10 showing the interaction between the connector and the rods comprising the spinal stabilizer of FIG. 9.

FIGS. 12 and 13 are alternative embodiments of transverse spinal stabilizers constructed in accordance with the teachings of the present invention.

### DETAILED DESCRIPTION OF THE PREFERRED EMBODIMENT(S)

In more detail, FIGS. 1-5 show a first embodiment of an apparatus for use in aligning and/or stabilizing the spinal column constructed in accordance with the present invention. Apparatus 10 is comprised of screw 12, screw cap 14, and extension 16, screw 12 being of a type known in the art that is comprised of an elongate threaded shank 18 and head 20. Screw cap 14 is comprised of a base 22 having a hook 24 extending therefrom, with means 26 formed in base 22 for retaining screw cap 14 to the head 20 of screw 12.

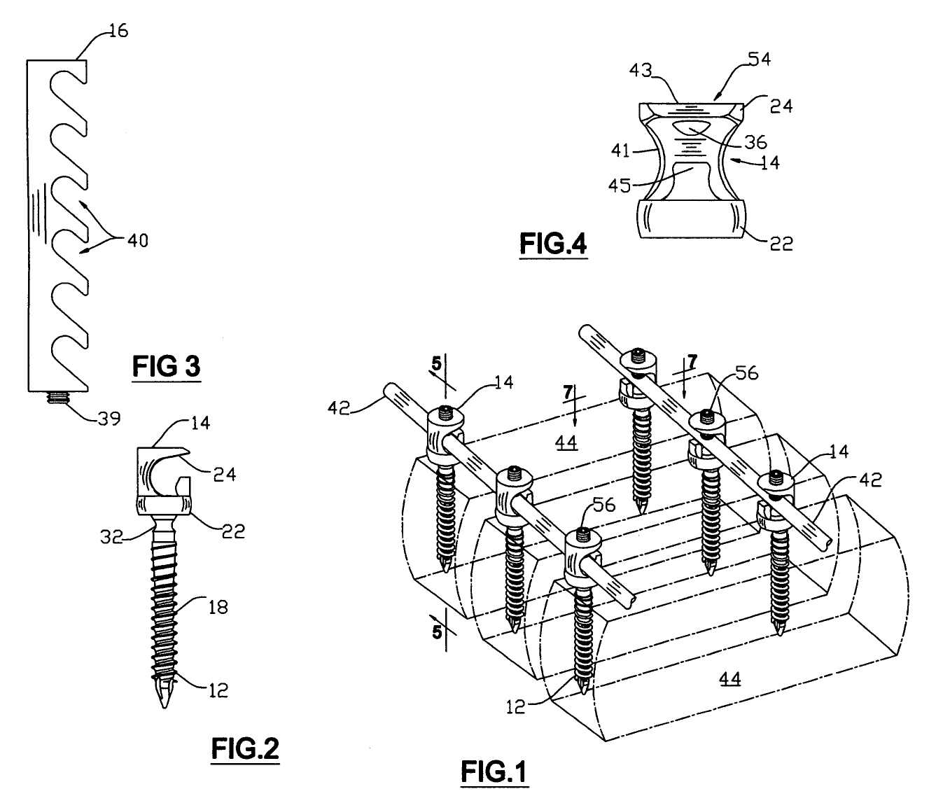

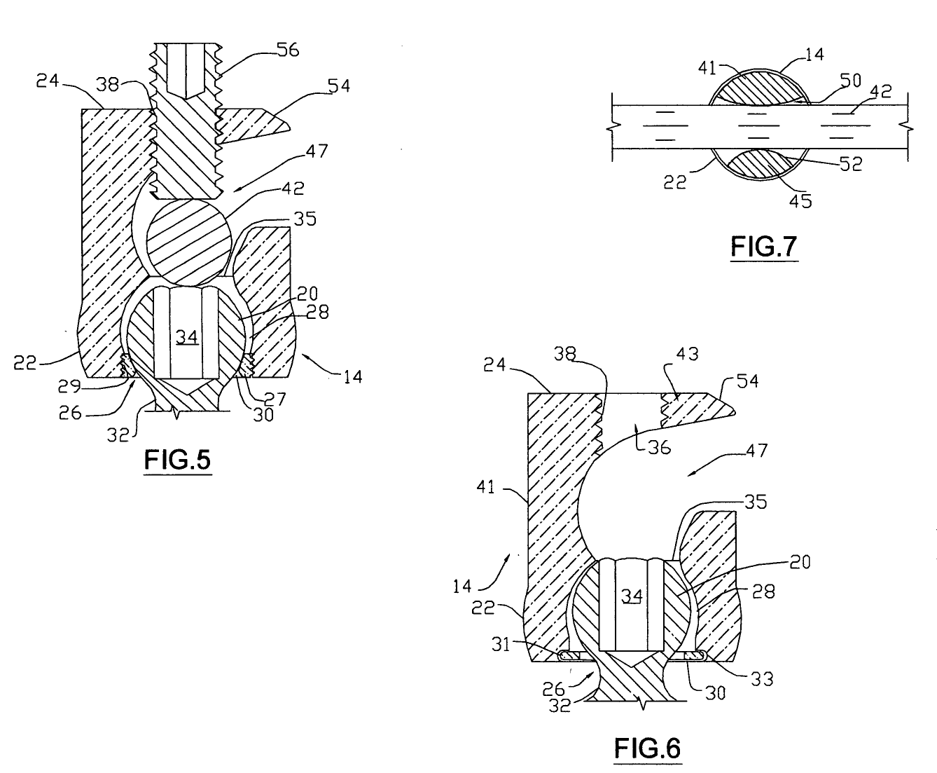

As shown in FIG. 5, screw cap retaining means 26 takes the form of a cavity 28 formed in the base 22 of screw cap 14, the cavity 28 being sized so as to receive the rounded head 20 of screw 12 therein while allowing the screw cap 14 to be positioned in an unlimited number of positions relative to screw 12 such that the cavity 28 and the head 20 of screw 12 function in a manner similar to a ball and socket joint. The ability to position screw cap 14 at an unlimited number of positions relative to screw 12 facilitates attachment of the rod of an external spinal stabilizer and/or apparatus for alignment of the spine to the screw 12 in the manner described below. In the embodiment shown in FIG. 5, screw cap retaining means 26 also comprises an “O”-ring 27 having screw threads 29 formed on the outside surface thereof that are threadably engaged to complimentary threads (not numbered) formed on the inside surface of the opening 30 into the cavity 28 in the base 22 of screw cap 14. This structure of screw cap retaining means 26 allows the head 20 of screw 12 to be retained in cavity 28 while the narrow neck 32 formed in screw 12 allows the screw cap 14 to be oriented at an unlimited number of positions relative to the long axis of screw 12 (in other words, the screw 12 and screw cap 14 need not be aligned on a common axis as shown in FIG. 2) to facilitate the connection between a rod and the screw 12 regardless of the angle between the long axis of screw 12 and the longitudinal axis of the spinal column, as well as the longitudinal axis of a rod such as the rod 42 shown in FIG. 1. Because screw cap retaining means 26 allows the screw cap 14 to be oriented at an unlimited number of positions, or angles, relative to the long axis of screw 12, the connection between rod 42 and screw 12 through screw cap 14 is referred to as being “polyaxial.”

An alternative embodiment of screw cap retaining means 26 is shown in FIG. 6. In the embodiment shown in FIG. 6, a split ring, or “C”-ring, 31 that is compressed until screw cap 14 is placed over the head 20 of screw 12. After the head 20 of screw 12 is positioned in cavity 28, the compressed split ring 31 is allowed to spring radially outwardly into the annular groove 33 formed in the wall of opening 30 to cavity 28. In the same manner that the threads 29 on “O”-ring 27 function to retain screw cap 14 to the head 20 of screw 12, the engagement of groove 33 by split ring 31, and the engagement of the inside margin of the hole (not numbered) through split ring 31 by the curved underside of the head 20 of screw 12 causes screw cap 14 to be retained to the head 20 of screw 12. Just as described in connection with the embodiment shown in FIG. 5, the narrow neck 32 of screw 12 allows the screw cap 14 to be oriented at an unlimited number of positions relative to the long axis of screw 12 (in other words, the screw 12 and screw cap 14 need not be aligned on a common axis as shown in FIG. 2) to facilitate polyaxial connection between rod and screw 12 as described above.

As best shown in FIGS. 5 and 6, the head 20 of screw 12 is provided with a bore 34 for receiving an instrument (not shown) for rotating screw 12 to drive screw 12 into the vertebral body. In the embodiment shown, bore 34 is shaped to receive a complimentary-sized hex key for rotating screw 12. Similarly, the hook 24 of screw cap 14 is provided with a bore 36 which serves a dual function, the first function being made possible by alignment of the axis of bore 36 with the axis of bore 34, which allows insertion of the hex key (not shown), or other instrument depending upon the shape of bore 34, for rotating screw 12 through bore 36 and into the bore 34 in the head 20 of screw 12. The second function of the bore 36 is enabled by the threads 38 formed in the walls of the bore, which threadably receive a threaded post 39 (not shown in FIGS. 5 and 6, see FIG. 3) on extension 16 (see FIGS. 1 and 3), thereby releasably securing extension 16 to screw cap 14. A third function of bore 34, as set out below, is to receive a set screw 56 for holding the rod in the cradle 47 (see below) formed in screw cap 14 to lock the rod 42 to the base 22 of screw cap 14. Although not immediately apparent from a review of FIG. 5 or 6 individually, by comparison of FIGS. 5 and 6, it can be seen that the head 20 of screw 12 projects through the opening 35 of cavity 28 into cradle 47 so that rod 42 pushes the head 12 into cavity 28 against the inside diameter of “O”-ring 27 (or the “C”-ring 31 described below in connection with FIG. 6) so as to lock the screw 12 at a particular angle (or at least resist relative motion) relative to rod 42. As is apparent from FIG. 5, when set screw 56 is tightened down against rod 42 to lock 42 against motion relative to screw cap 14, and when formed so as to receive a hex key as shown in the preferred embodiment in FIG. 5, the shape of bore 34 functions to help resist relative motion between rod 42 and screw 12 because the outside diameter of rod 42 is forced against the top of the head 20 of screw 12 against the top margin, or opening, of bore 34 such that the margins of the opening function to provide a bearing surface for the surface of rod 42.

Referring now to FIG. 4, as well as FIGS. 5 and 6, it can be seen that the portion of screw cap 14 that comprises hook 24 is formed of an upright riser 41 and an overhanging portion 43, the threaded bore 36 being formed in the overhanging portion 43 of hook 24. As best shown in FIG. 4, in addition to screw cap retaining means 26, the base 22 of screw cap 14 comprises a post 45. As shown in FIGS. 1 and 5, the hook 24 of screw cap 14 is adapted for receiving and retaining a rod 42 to the screw cap 14, and to facilitate that function, the riser 41 of hook 24, overhanging portion 43 of hook 24, and post 45 of base 22 together define a cradle, indicated at reference numeral 47 in FIGS. 5 and 6, for receiving rod 42. As described above, the screws 12 in adjacent segments often project from the vertebral body at different angles and, although the ball and socket joint formed between the head 20 of screw 12 and screw cap 14 accommodates some of that variability, the screws in adjacent segments commonly do not line up, especially when the apparatus of the present invention is utilized for aligning a congenitally mis-aligned, damaged, or diseased spine such that rod 42 does not pass at a right angle through the cradle 47 in screw cap 14 at a particular segment. To accommodate this additional variability, the surface of riser 41 of hook 24 adjacent rod 42 when the rod is retained in cradle 47 is formed in a convex shape as shown at reference numeral 50 (see FIG. 7). For the same reason, the surface of post 45 is also formed in a convex shape as shown at reference numeral 52 (also best shown in FIG. 7).

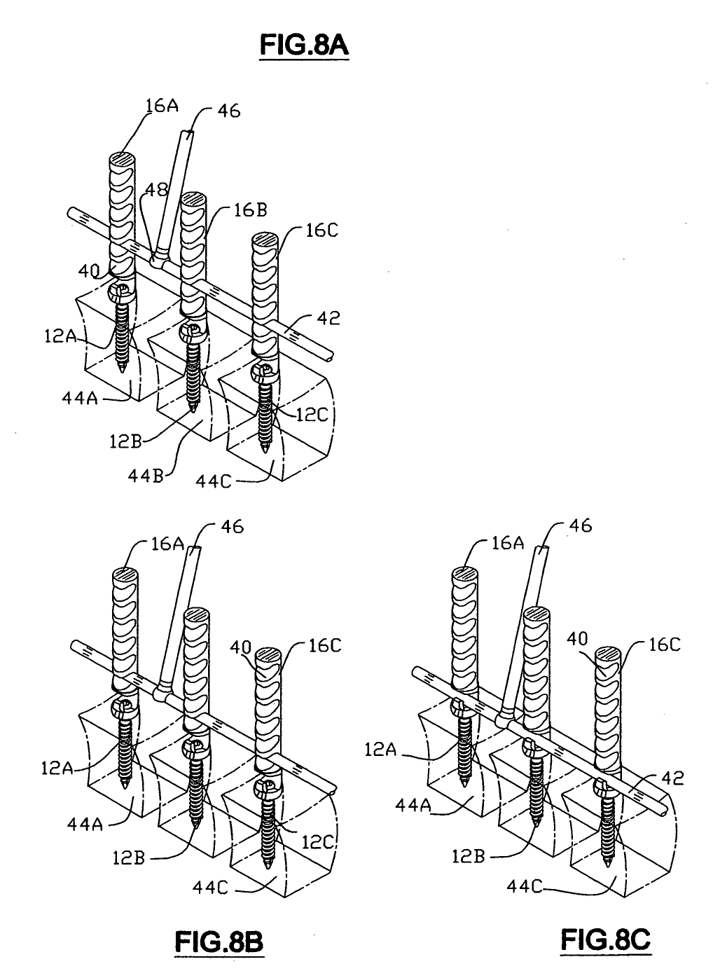

Referring back to FIG. 3, extension 16 is provided with a plurality of notches 40, six such notches 40 being shown in FIG. 3 (and in each of FIGS. 8A, 8B, and 8C), forming a ladder that is adapted for releasably retaining a rod 42 thereto as shown in FIGS. 8A, 8B, and 8C (those skilled in the art who have the benefit of this disclosure will recognize that the number of notches 40 formed on extensions 16 is a matter of choice and/or selection based upon the needs of an individual patient). Referring to FIG. 8A, three screws 12A, 12B, and 12C are shown driven into respective vertebral bodies shown schematically at reference numerals 44A, 44B, and 44C in each of FIGS. 8A, 8B, 8C, and the respective screw caps 14A, 14B, and 14C are retained to each screw. After the corresponding extensions 16A, 16B, and 16C have been threaded onto the respective screw caps 14A, 14B, and 14C, the rod 42 is pushed downwardly toward the heads 20A, 20B, and 20C of screws 12A, 12B, and 12C at different locations along rod 42 proximate each extension 16A, 16B, and 16C using a suitable instrument such as is shown at reference numeral 46 having a fork 48 or other similarly-shaped tip formed thereon to facilitate manipulation of rod 42. In this manner, rod 42 is positioned in one of the notches 40 formed on each of the respective extensions 16A, 16B, and 16C, FIG. 8A showing the rod 42 positioned in the third lowest notch 40 in each extension 16. Using the instrument 46, the rod is then pushed downwardly to slide rod 42 out of the third lowest notch 40 (FIG. 8A) and manipulated so as to slip into the next lower notch 40 (FIG. 8B), e.g., from the “third rung” on the ladder formed by the notches 40 on extension 16 to the second rung of that ladder. Because of the above-described difficult geometry of the connection between the rod and a pedicle screw, the rod may be only loosely positioned in a first notch such as is shown in FIG. 8A and then, as the process of pushing the rod 42 out of the notch and down the ladder to the next lower notch 40 is repeated at various points along the length of rod 42 with the rod 42 fitting tighter and tighter at each rung of the ladder until the rod is positioned in the lowest notch 40 on extension 16 in FIG. 8C.

From the lowest notch 40 (shown in FIG. 8C), rod 42 is pushed downwardly again, but in this last step, the rod is slipped under the overhanging portion 43 of hook 24 formed on screw cap 14 and into the cradle 47. In this manner, the rod 42 is gradually worked down the ladder to a position in which the load of the spinal column is effectively transferred from the spinal column to rod 42 and/or the adjacent vertebrae 44A, 44B, and 44C are aligned with each other and retained in alignment by the transfer of load through rod 42. To facilitate the working of rod 42 down the ladder formed by the notches 40 in extension 16 and into the cradle 47 of screw cap 14A, 14B, and 14C, which is likely to be the position in which rod 42 is tightest and therefore most difficult to “steer” into the cradle 47 with instrument 46, the leading edge of the overhanging portion 43 of hook 24 is beveled as shown at reference numeral 54 (see FIGS. 4, 5, and 6).

As shown in FIG. 1, after the rod 42 has been pushed downwardly into the cradle 47 of screw cap 14, the extensions 16A, 16B, and 16C are removed from the corresponding screw caps 14A, 14B, and 14C. A set screw 56 is then threaded into the same bore 36 from which each of the extensions 16 have been removed and tightened against the rod 42 to retain the rod 42 under the hook 24 of screw cap 14 and further to lock or resist movement of the screw head 20 relative to the base 22 of screw cap 14 in the manner described above in connection with the description of FIG. 5.

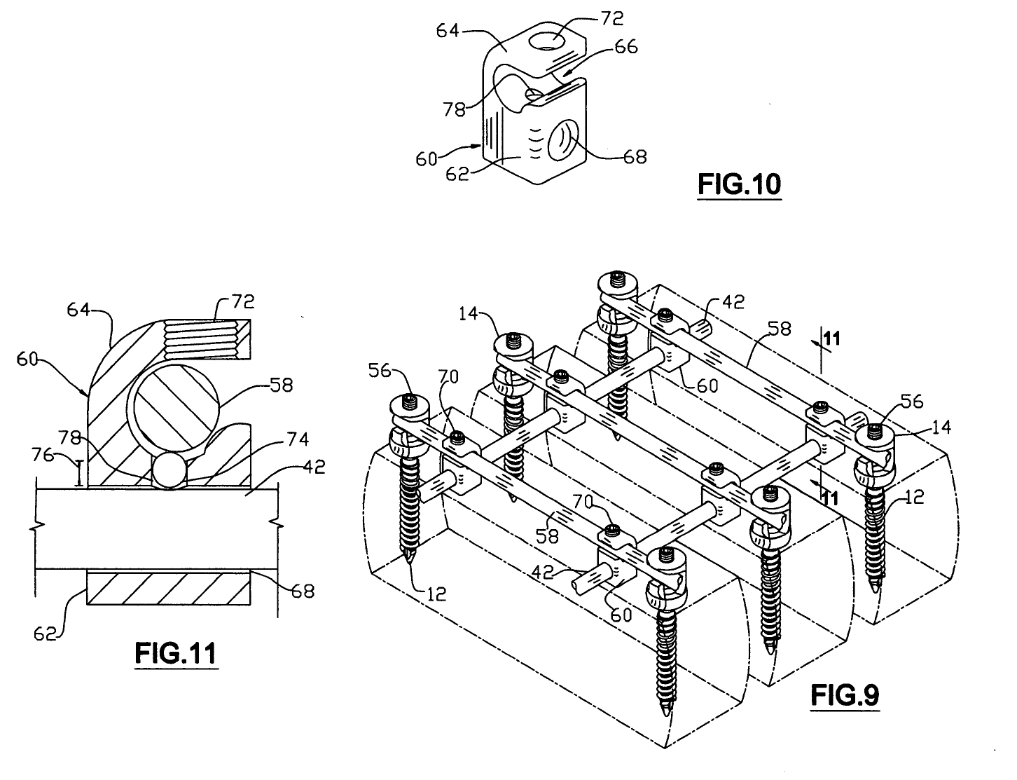

The apparatus of the present invention has the additional advantage of being adaptable for use in transverse stabilization of the spinal column as shown in FIG. 9. As is apparent from a comparison of FIG. 9 to FIG. 1, in the transverse system shown in FIG. 9, the screw caps 14 retained to the pedicular screws 12 mounted bilaterally on vertebral bodies 44 are rotated approximately 90° relative to the longitudinal axis of the spinal column for receipt of a transverse rod 58 in the respective cradles 47 thereof (by comparison, it can be seen that the axis of the cradle 47 formed in the screw caps 14 is substantially parallel to the longitudinal axis of the spinal column in FIG. 1). Each transverse rod 58 is provided with two connector blocks 60, and as best shown in FIGS. 10 and 11, each connector block 60 comprises a base 62 and overhanging portion 64 that together form a cradle 66 in a manner similar to the cradle 47 formed by the base 22 and overhanging portion 24 of the screw caps 14. Connector block 60 also comprises a horizontal bore 68 that extends through the base 62 of connector block 60 at an angle that is substantially orthogonal to the axis of cradle 66 for receipt of a rod 42 that is aligned with the longitudinal axis of the spinal column of the patient. Although two substantially parallel, longitudinally-extending rods 42 are shown in FIG. 9, those skilled in the art who have the benefit of this disclosure will recognize that the apparatus of the present invention is also adaptable for use in a system including just a single longitudinally-extending rod 42 depending upon the particular pathology of the patient.

Each connector block 60 is provided with a set screw 70 extending through a threaded bore 72 in the overhanging portion 64 of the connector block that is tightened against the outer diameter of the transverse rod 58 to which the respective connector block 60 is mounted. As shown in FIG. 11, connector block 60 also comprises a ball bearing 74 that is captured in the vertically-extending bore 76 formed in the base 62 of connector block 60 between the transverse rod 58 and the longitudinally-extending rod 42. Ball bearing 74 is slightly larger in diameter than the vertical dimension 76 of the vertically-extending bore 78 so that the top and bottom surfaces of ball bearing 74 extend out of the top and bottom of the vertically-extending bore 78 with the result that, when set screw 70 is tightened against the outer diameter of transverses rod 58, the surface of transverse rod 58 bears against the top surface of ball bearing 74, pushing ball bearing 74 downwardly in vertically-extending bore 78 and into contact with the outer diameter of longitudinally-extending rod 42. In this manner, set screw 70 functions to retain connector block 60 in position on both transverse rod 58 and longitudinally-extending rod 42. Of course those skilled in the art will recognize that this construction enables connector block 60 to be positioned along the length of both the longitudinally-extending rod 42 and the transverse rod 58 during the surgical procedure as required by the anatomy and/or the pathology of a particular patient while still allowing the surgeon to tighten the set screw 70 to resist movement of connector block 60 relative to either of the rods 42 and/or 58.

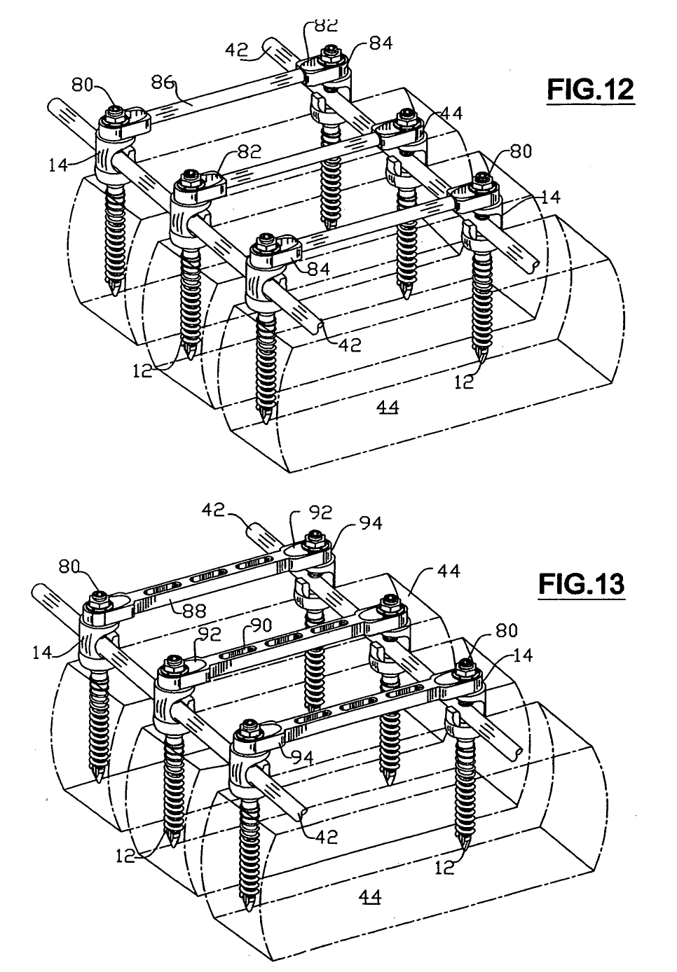

Referring now to FIGS. 12 and 13, it can be seen that the apparatus of the present invention is also adaptable for use with transverse rods of a type other than the rod 58 shown in FIG. 9. For instance, as shown in FIG. 12, the set screw 56 shown in FIG. 1 is replaced by a threaded rod 80 that is tightened against the longitudinally-extending rod 42 in the same manner as described above in connection with the embodiment shown in FIG. 1 but which extends vertically to a height sufficient to provide a post that projects through the slots 82 formed in the ends 84 of a transverse bar 86. A locking nut 88 is then tightened down against the margins of the slots 82 at either end of transverse bar 86 to resist movement of bar 86 relative to the vertebrae 44. Referring to FIG. 13, the transverse bars 88 are of a type known in the art having a plurality of longitudinally-extending slots 90 formed along the length of the bar in addition to the slots 92 formed at the ends 94 of bar 88.

Those skilled in the art who have the benefit of this disclosure will recognize that certain changes can be made to the component parts of the apparatus of the present invention without changing the manner in which those parts function and/or interact to achieve their intended result. By way of example, those skilled in the art who have the benefit of this disclosure will recognize that screw cap retaining means 26 may be modified in any of several ways but still function to achieve the same result. For instance, rather than the “O”-ring 27 or “C”-ring 31 that are assembled to the base 22 of screw cap 14 in the opening 30 in the bottom of the screw cap as shown in FIGS. 5 and 6, a “U”-shaped bracket, or keeper, could be inserted into a complimentary-shaped opening in the side of screw cap 14 so that the arms of the “U” slip under the head 20 of screw 12 above the neck 32 of the screw. The arms of a “U”-shaped keeper constructed in accordance with the present invention are preferably provided with outwardly extending prongs or keys that spring outwardly into complimentary-shaped recesses in the opening in the side of screw cap 14 so as to retain the keeper in the screw cap. Other structure that functions to achieve this same result will be apparent to those who have the benefit of this disclosure. It will also be recognized by those skilled in the art that, rather than the connector blocks 60 shown in FIGS. 9-11, the transverse and longitudinal rods and/or bars of the apparatus of the present invention can be connected by structure formed in the shape of back-to-back cradles such as the cradles 47 and 66 with set screws such as the set screws 56 and 70 extending at the appropriate angles so that the rods can be set in place as needed during the surgical procedure. Similarly, each of the notches 40 forming the ladder on extension 16 may be provided with a beveled surface to facilitate the re-positioning of the rod 42 in successive notches, or rungs of the ladder, such as the surface 54 formed on the overhanging portion 43 of the hook 24 of screw cap 14. All such changes, and others that will be clear to those skilled in the art from this description of the preferred embodiments of the invention, are intended to fall within the scope of the following, non-limiting claims.

What is claimed is:

1. Apparatus for use in aligning and/or stabilizing the spinal column comprising:
a screw comprised of an elongate, threaded shank and a head;
a screw cap comprised of a base having a hook extending therefrom, the hook being adapted for retaining a rod to said screw cap;
means formed in the base of said screw cap for retaining said screw cap to the head of said screw at any of a plurality of angles relative to the long axis of said screw;
an extension having a plurality of notches formed thereon, each of the notches being adapted for releasably retaining a rod to said extension; and
means formed on said extension for releasably securing said extension to said screw cap.

2. The apparatus of claim 1 wherein said means for releasably securing said extension to said screw cap comprises a threaded post integral with said extension and a threaded bore formed in the hook of said screw cap for receiving the threaded post.

3. The apparatus of claim 2 wherein the head of said screw is provided with a bore for receiving an instrument for turning said screw.

4. The apparatus of claim 3 wherein the axis of the bore in the head of said screw is substantially aligned with the axis of the bore in the hook of said screw cap.

5. The apparatus of claim 2 additionally comprising a set screw adapted for tightening in the bore formed in the hook of said screw cap.

6. The apparatus of claim 1 wherein the hook of said screw cap is comprised of an upright portion and an overhanging portion, the upright portion being rounded on the surface adjacent a rod when retained to said screw cap.

7. The apparatus of claim 6 additionally comprising a bore formed in the overhanging portion of said screw cap for receiving an instrument for turning said screw.

8. The apparatus of claim 1 additionally comprising a riser formed on the base of said screw cap.

9. The apparatus of claim 8 wherein the surface of said riser adjacent a rod when retained to said screw cap is concave-shaped.

10. The apparatus of claim 1 wherein said screw cap retaining means comprises a cavity for receiving the head of said screw therein.

11. The apparatus of claim 10 wherein said screw cap retaining means additionally comprises a ring assembled to said screw cap under the head of said screw.

12. The apparatus of claim 11 wherein said ring is provided with threads formed on the outside surface thereof for threadably engaging complimentary threads formed on the inside surface of the cavity formed in said screw cap.

13. The apparatus of claim 12 wherein said ring is a split ring that is compressed for insertion into the cavity formed in said screw cap and for bearing against the inside wall of the cavity when allowed to expand.

14. A method of aligning the spinal column comprising the steps of:
driving a plurality of bone screws, the head of each bone screw being provided with a screw cap comprised of a base and a hook, into the bodies of a like number of spinal vertebrae;
affixing an extension to each of the bone screws, each extension having a plurality of notches adapted for releasably retaining a rod formed thereon;
inserting a rod running substantially parallel to the spinal column into a notch on each extension;
pushing the rod out of the notch and downwardly toward the head of the bone screw either into a next lower notch or until the rod is pushed downwardly under the hook of the screw cap; and
removing the extension from the screw cap.

15. The method of claim 14 wherein each of the bone screws is rotated to drive the screw into the body of the spinal vertebrae using an instrument inserted into the head of the screw through a hole in the hook of the screw cap.

16. The method of claim 15 additionally comprising securing the rod beneath the hook of the screw cap.

17. The method of claim 16 wherein the rod is secured beneath the hook of the screw cap by means extending through the hole in the hook of the screw cap.

18. The method of claim 14 wherein the extension is affixed to the screw cap by structure extending into the hole in the hook of the screw cap.
